(19) United States
(12) Patent Application Publication
(10) Pub. No.: US 2016/0374578 A1
(43) Pub. Date: Dec. 29, 2016

(54) CONTEXTUAL HEART HEALTH MONITORING WITH INTEGRATED ECG (ELECTROCARDIOGRAM)

(71) Applicant: Intel Corporation, Santa Clara, CA (US)

(72) Inventors: Ray Kacelenga, Hillsboro, OR (US); Uttam K. Sengupta, Portland, OR (US)

(21) Appl. No.: 14/749,847

(22) Filed: Jun. 25, 2015

(51) Int. Cl. A61B 5/0404; A61B 5/00; A61B 5/0488; A61B 5/0245; A61B 5/0408

(52) U.S. Cl. CPC A61B 5/0404; A61B 5/0245; A61B 5/04085; A61B 5/0488; A61B 5/6898; A61B 2562/0219

(57) ABSTRACT

Integrated ECG (electrocardiogram) contacts enable opportunistic heart rate monitoring on a handheld electronic device. First and second ECG contacts are integrated into the device to connect, respectively, first and second ECG electrodes to an internal ECG circuit within the device. The ECG electrodes have vertical and horizontal portions that can be separate portions connected to a common contact, or different portions of an ‘L-shaped’ electrode. The ECG electrodes are positioned on opposite sides of the device to enable opportunistic two-hand contact when the device is used in either landscape or portrait orientation. The internal ECG circuit is to detect two-hand contact by the user on the first and second electrodes, and perform ECG monitoring in response to detecting two-hand contact. A mobile device can opportunistically capture heart rate data along with user context and provide alerts if a deviation is detected between heart rate data and user activity.

### FIELD

Embodiments of the invention are generally related to sensors integrated on mobile device, and more particularly to contextual heart health monitoring via ECG sensors integrated on a mobile device.

### COPYRIGHT NOTICE/PERMISSION

Portions of the disclosure of this patent document may contain material that is subject to copyright protection. The copyright owner has no objection to the reproduction by anyone of the patent document or the patent disclosure as it appears in the Patent and Trademark Office patent file or records, but otherwise reserves all copyright rights whatsoever. The copyright notice applies to all data as described below, and in the accompanying drawings hereto, as well as to any software described below: Copyright© 2015, Intel Corporation, All Rights Reserved.

### BACKGROUND

Health and wellness enthusiasts who track their vitals during workout sessions want to log their Heart Rate (HR) not only during a workout session but also in between workout sessions. Such HR monitoring is best achieved by wearing a HR monitor either on the wrist or chest, with the data sent over to a mobile device (e.g., smartphone, tablets, or other devices) for analysis. The analysis can be performed on the local mobile device, or via cloud services. HR monitors have also been added to ear buds to enable continuous monitoring of Heart Rate.

Because of the increased interest in HR monitoring, some smartphone vendors add HR monitors to their devices. Some smartphones use photoplethysmography (PPG) signals using pulse oximetry. A pulse oximeter illuminates a wearer's skin using a light emitting diode (LED) and measures intensity changes in the light reflected from skin and finger tissue, forming a PPG signal. The periodicity of the PPG signal corresponds to the cardiac rhythm, and thus, heart rate can be estimated using the PPG signal. However, the HR estimation requires the user to hold their finger in place for several seconds (30 seconds or more), while holding still and not talking as the monitor calculates the HR.

PPG-based HR sensors are not considered to be as accurate as ECG (electrocardiography). ECG sensors directly use electrical signals produced by heart activity whereas PPG uses electrical signals derived from light reflected due to changes in blood flow during heart activity. In addition to being measured more accurately, ECG sensors do not require long settling times, which allows meaningful readings to be obtained faster than PPG sensors.

Smartphone vendors would typically prefer the improved accuracy and faster settling times for ECG sensors. Smartphone designs that incorporate ECG sensors include electrodes placed on the back cover of the devices. The use of the ECG capability traditionally requires very deliberate action by the user. The user must open a specific application on the device, and stop, stand still, and hold the device. Additionally, traditional ECG capability is used exclusively in portrait or landscape mode. Thus, in the other mode (portrait or landscape), the ECG waveform is unobservable and the logging capability is absent.

### BRIEF DESCRIPTION OF THE DRAWINGS

The following description includes discussion of figures having illustrations given by way of example of implementations of embodiments of the invention. The drawings should be understood by way of example, and not by way of limitation. As used herein, references to one or more “embodiments” are to be understood as describing a particular feature, structure, and/or characteristic included in at least one implementation of the invention. Thus, phrases such as “in one embodiment” or “in an alternate embodiment” appearing herein describe various embodiments and implementations of the invention, and do not necessarily all refer to the same embodiment. However, they are also not necessarily mutually exclusive.

FIG. 1 is a block diagram of an embodiment of a system that performs opportunistic heart rate monitoring.

FIG. 2A is a block diagram of an embodiment of a computing device with strategically placed ECG electrodes for opportunistic heart rate monitoring.

FIG. 2B is a block diagram of an embodiment of a computing device with strategically placed ECG electrodes for opportunistic heart rate monitoring.

FIG. 3 is a block diagram of an embodiment of a device cover having strategically placed ECG electrodes for opportunistic heart rate monitoring.

FIG. 4 is a block diagram of an embodiment of a system that performs opportunistic monitoring including detecting whether user contact is two-handed or one-handed.

FIG. 5A is a diagrammatic representation of an embodiment of a one-handed ECG signal.

FIG. 5B is a diagrammatic representation of an embodiment of a two-handed ECG signal.

FIG. 6 is a flow diagram of an embodiment of a process for monitoring an ECG input.

FIG. 7 is a flow diagram of an embodiment of a process for opportunistic heart rate monitoring.

FIG. 8 is a block diagram of an embodiment of a mobile device in which opportunistic heart rate monitoring can be implemented.

Descriptions of certain details and implementations follow, including a description of the figures, which may depict some or all of the embodiments described below, as well as discussing other potential embodiments or implementations of the inventive concepts presented herein.

### DETAILED DESCRIPTION

As described herein, integrated ECG (electrocardiogram, also referred to as EKG from the term “elektrokardiogram”) contacts enable opportunistic heart rate monitoring on a handheld electronic device. First and second ECG contacts are integrated into the device to connect, respectively, first and second ECG electrodes to an internal ECG circuit within the device. The ECG electrodes have vertical and horizontal portions that can be separate portions connected to a common contact, or different portions of an ‘L-shaped’ electrode. The ECG electrodes are positioned on opposite sides of a face of the body of the device to enable opportunistic two-hand contact by a user of the device when the device is used in either landscape or portrait orientation. The internal ECG circuit is to detect two-hand contact by the user on the first and second electrodes, and perform ECG monitoring in response to detecting two-hand contact.

The integration of the ECG electrodes enables a mobile device to include ECG capability that can measure heart rate (HR) information accurately without long settling times or requiring compensation of motion artifacts to produce a reading. The integration of the electrodes is in the body of the handheld computing device (e.g., smartphone, tablet). The integration in the body of the device can be directly in the housing that makes up the device and/or in a cover that connects to contacts in the body of the housing of the device. ECG requires two-handed operation, with each hand touching one of the opposing electrodes. The electrodes described herein enable opportunistic contact by the user of the opposing electrodes. By each electrode having horizontal and vertical portions, the device can obtain an opportunistic ECG reading whether the device is used in landscape or portrait mode.

In one embodiment, the opportunistic monitoring enables the correlation of HR information with other sensor information to provide contextual use of the HR information. For example, readings from motion, environmental, and/or other sensors in addition to the ECG can put the heart rate information in the user's context. Thus, for example, the system can correlate HR readings with the user's context (e.g., walking, running, talking, browsing/reading) because it does not require the user to change context. In one embodiment, the system can capture the data dynamically, in the background without any user intervention.

As described herein, the placement of the ECG electrodes enables more spontaneous and opportunistic HR data monitoring than traditional methods, and does not require the user to change context or behavior to obtain a heart rate reading. The opportunistic placement of the ECG electrodes increases the probability of user contact with the electrodes during regular daily interaction with the handheld device. The user does not need to be cognizant of where the electrodes are located. Furthermore, the user does not need to be compelled by an application running on the host platform of the device to adopt prescribed postures and engage in a scripted procedure for acquiring an ECG signal. In one embodiment, the device automatically captures an ECG signal whenever Left (L) and Right (R) electrodes are opportunistically touched by the left and right hand appendages (finger, thumb, palm), respectively, and remain in contact for a specified minimum duration.

It will be understood that an ECG or EKG sensor measures the natural electrical activity of the heart when the heart is pumping blood to the lungs and the rest of the human body. In general, an ECG sensor includes electrodes placed to be connected to left and right sides of the body, to form a closed loop circuit through the user and through the ECG circuit. The ECG sensor includes analog differential amplifiers that detect, filter, amplify, and condition the small electrical signals generated as the heart beats. Backend digital filters (e.g., notch filters) are typically employed to remove 50 Hz and 60 Hz mains interference. The ECG waveform is typically used for a variety of health assessments such as detecting atrial fibrillation, arrhythmias, anginas, and other heart anomalies. As described herein, a contextual system can use the ECG waveform for determining fitness and wellness parameters such as Heart Rate (beats/minute), stress monitoring, and mood analysis. In addition, the ECG waveform can be used for user authentication or personalization use cases.

As described herein, opportunistic HR monitoring from strategically placed electrodes on a handheld electronic device can enable a number of different use cases based on contextual ECG or contextual HR information. For example, a system can enable stress management. Heart rate variability information (HRV) opportunistically recorded from the ECG contacts can be a stress indicator. Application of the HRV information can enable short term and long term stress measurement and tracking, as well as trending information (whether average HRV is increasing or decreasing over time) based on smoothed stress data. Application of the HRV information can enable a handheld device to determine emotional state of the user. Detection techniques are known for emotions such as frustration, calmness, appreciation, anger, and focus. The HRV information can be used to log the frequency of detected emotions over time (e.g., weeks or months). The HR information can be used to estimate Heart Age, which is a measure of heart health as compared to physical age. Such information can provide a running plot of Heart Age over time (e.g., weeks or months). In one embodiment, the HR information can be used for bio-identification and authentication. For example, a handheld device can discriminate between device users based on ECG signatures, and/or use ECG signature to log in to secure portals.

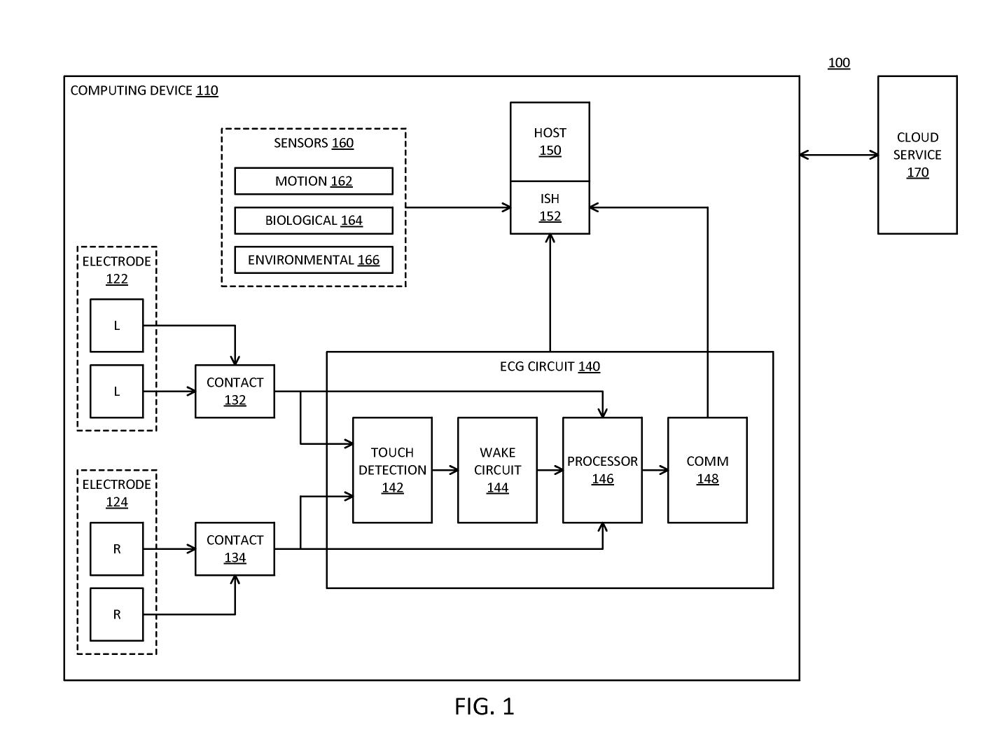

FIG. 1 is a block diagram of an embodiment of a system that performs opportunistic heart rate monitoring. System 100 includes handheld computing device 110. Device 110 can be, for example, a tablet, a smartphone, or other electronic computing device that is used in the hands. There are specialized devices (such as wrist-based devices (watches or bands)) that monitor HR and movement. However, such a device is separate from a computing device that a user might otherwise own and use regularly. Additionally, the handheld computing devices can provide access to contextual processing that a fitness accessory typically cannot provide.

Thus, device 110 allows a user to log the user's ECG/HR measurements without being consciously involved in any way beyond normal usage of the computing device. Device 110 includes ECG circuit 140 integrated into the device. For example, ECG circuit 140 can include an application specific integrated circuit (ASIC) and/or other logic built into the hardware platform of device 110. In one embodiment, ECG circuit 140 includes touch detection 142, wake circuit 144, processor 146, and communication (comm) 148. ECG circuit 140 can interface with ECG electrodes 122 and 124, respectively, via contacts 132 and 134.

ECG circuit 140 includes an analog front end (AFE) to interface with ECG electrodes. The AFE can include touch detection circuitry 142, which enables ECG circuit 140 to determine when there is a closed circuit via contact with the electrodes. Wake circuit 144 enables ECG circuit 140 to keep processor 146 and communication 148 in a low power state while there is no ECG input to process. Such a low power state can provide significant power savings when using the device. Wake circuit 144 can include signal detection hardware (e.g., such as a preprocessor) to detect an ECG signal of interest, and generate a wake signal or enable signal to processor 146 in response to detecting the ECG signal.

Electrodes 122 and 124 are integrated onto computing device 110. In one embodiment, specific electrodes are placed as “left-hand” electrodes and others as “right-hand” electrodes, as illustrated respectively with electrodes 122 and 124. Electrodes 122 and 124 are differential electrodes in that an AFE can generate a signal as a difference between the two electrodes. In one embodiment, each electrode includes at least a horizontal and a vertical portion. The separate portions can be separate conductive surfaces that tie to a common contact. For example, as illustrated, all contact surfaces for electrode 122 can connect to contact 132, and all contact surfaces for electrode 124 can connect to contact 134. The electrodes can include two or more surfaces coupled to the common contact. Contact with any one or more surface of each electrode can provide a closed loop for HR monitoring. In one embodiment, the electrodes have a single surface that has an “L” shape, where horizontal and vertical portions are connected to each other. In one embodiment, each electrode has two or more separate portions where the contact surface are not connected to each other on the body of the device, but are electrically coupled to the same contact. In one embodiment, electrodes 122 and 124 are integrated into a housing of device 110. In one embodiment, electrodes 122 and 124 are integrated into a housing of a cover of device 110, and are designed to contact contacts 132 and 134 integrated into the housing of device 110.

In one embodiment, processor 146 is a mixed signal processor, which receives analog inputs from contacts 132 and 134 and processes the signals. Communication 148 represents hardware that enables ECG circuit 140 to provide HR information to a host processor for contextual processing. Communication 148 can include a UART (universal asynchronous receiver-transmitter), I²C (inter-integrated circuit) interface, SPI (system programming interface), BLE (Bluetooth low energy), and/or other communication hardware. In one embodiment, ECG circuit 140 includes other components not specifically identified in system 100.

Device 110 includes host 150, which represents a host processor or processing core for device 110. Host 150 is a processor that executes a host operating system for device 110. The host operating system controls the functions and the flow of operation of the device as a whole. Processor 146 controls the operation of ECG circuit 140 and provides the resulting HR information to host 150. In one embodiment, ECG circuit 140 is coupled to host 150 via a sensor hub, such as ISH (integrated sensor hub) 152. In one embodiment, ISH 152 is integrated into host 150, and can be a circuit that is part of a processor die and/or part of a processor system on a chip (SoC). ISH 152 can manage data access and control of various sensors in device 110.

In one embodiment, device 110 includes multiple sensors 160 in addition to the ECG sensor of ECG circuit 140. Sensors 160 can include one or more sensors of one or more sensor types. Types of sensors can include motion sensors, biological sensors, environmental sensors, and/or others. In one embodiment, sensors 160 include motion sensors 162, which can include accelerometers, positioning units, and/or other sensors. In one embodiment, sensors 160 can include biological sensors 164, which can include other sensors to track biological information for a user. In one embodiment, sensors 160 can include environmental sensors 166, which can include temperature sensors, audio sensors, light sensors, and/or other sensors.

In one embodiment, ISH 152 receives information from ECG circuit 140 as well as one or more other sensors 160. Host 150 can generate contextual information from the received sensor data. For example, host 150 can determine that certain HR values are associated with movement of the user, or fluctuations in HR can occur as a result of music being played on the device, or by emails or other communication received/sent via the device, or other contextual information. In one embodiment, host 150 provides HR information, which can be raw HR information and/or contextual HR information, to cloud service 170. The portion of host 170 that connects to cloud service 170 can include a host processor and/or other logic and hardware on device 110. Cloud service 170 represents a processing resource external to device 110 that is accessed via a network communication link (e.g., WiFi, cellular, or other). In one embodiment, cloud service 170 provides analysis of HR information, and can trigger alerts or other messages to a user from opportunistically measured HR data.

Thus, in one embodiment, HR/ECG information logged by ECG circuit 140 can be interpreted at host 150 and/or at cloud service 170 in a frame of reference provided by other device context sources such as physical activity and the prevailing ambient environmental factors as indicated by other sensors 160. In one embodiment, ECG circuit 140 opportunistically monitors HR information and provides it for use to host 150, which can include the operating system and any applications executing on the host. ECG circuit 140 can therefore gather HR information without requiring the user to invoke an application or performing a deliberate action. Such opportunistic measurements can enable dynamic user contexts to be captured to provide a reference frame for interpreting the measured ECG/HR information.

Consider the following user case scenarios for device 110, in which ECG circuit 140 and host 150 can provide contextual responses to HR information. In one example, a user composes email with device 110 using two hands on the device. The user can hold device 110 in either portrait or landscape mode, and can be sitting down, or walking around. The user touches both hands opportunistically on respective electrodes and triggers ECG circuit 140. In one embodiment, ECG circuit 140 asserts an interrupt signal to ISH 152. ISH 152 logs the ECG data stream transmitted via communication 148, along with motion, location, environmental, and/or biological context data from sensors 160. In one embodiment, ISH 152 or host 150 includes fusion algorithms to determine that the user's HR, derived from ECG circuit 140, is 77 BPM (beats per minutes) and is higher than the resting (baseline) value of 66 BPM for the user. The fusion algorithm then determines that the user is actually walking and that the elevated HR is consistent with the user walking; thus, the rise in HR is expected.

In a second example, consider that the user runs up the stairs to a fifth floor apartment. When the user arrives at the apartment, huffing and puffing and out of breath, the user pulls out device 150 to catch up on social media, news, or sports. Both hands opportunistically rest on the respective electrodes and trigger ECG circuit 140, which can assert an interrupt signal to ISH 152. In one embodiment, ISH 152 or host 150 logs the ECG data stream transmitted via communication 148, along with motion, location, environmental, and biological context data from sensors 160. Algorithms in ISH 152 or host 150 determine that the user's HR, derived from ECG circuit 140, is 145 BPM and is higher than the resting (baseline) value of 66 BPM for the user. The fusion algorithm then determines, from context history derived from data from sensors 160, that the user just ran up 5 floors. Thus, the algorithms determine that the elevated HR is consistent with strenuous physical activity and is to be expected.

In a third example, consider a user is building a house, and the developer gives the user only a few hours to select both internal and external colors for the house. The user pulls out device 110 and furiously starts looking at house colors on several websites. Both hands opportunistically rest on the respective electrodes and trigger ECG circuit 140, which can assert an interrupt signal to ISH 152. In one embodiment, ISH 152 or host 150 logs the ECG data stream transmitted via communication 148, along with motion, location, environmental, and biological context data from sensors 160. ISH 152 or host 150 includes a Heart Rate Variability (HRV) algorithm, and determines from the algorithm that the user's HR is elevated and that the HRV power spectrum is dominated by very low frequencies (VLF). A calculation of a Coherence Ratio reveals a very low coherence of 0.2. Based on these findings, the fusion algorithm determines that the user is anxious or stressed, and triggers a software function to provide an alert to the user. The software function (e.g., a process or service executing on the host operating system or a separate application running under the operating system) generates an alert to the user, recommending a breathing regimen of 5 seconds inhalation and 5 seconds exhalation for 5 minutes to unwind and declutter the cognitive centers of the brain.

In a fourth example, consider an elderly user who lives in an area that has seen a significant amount of snowfall. Temperatures have fallen precipitously to historic lows, and the user decides to bundle up to go shovel the snow off the driveway. After two or so hours of shoveling in the bitter cold, the user comes back into the house and heads straight for the gas furnace to warm up. The user picks up device 110 to check the weather forecast for the following day. Both hands opportunistically rest on the respective electrodes and trigger ECG circuit 140, which can assert an interrupt signal to ISH 152. In one embodiment, ISH 152 or host 150 logs the ECG data stream transmitted via communication 148, along with motion, location, environmental, and biological context data from sensors 160. HRV algorithms on ISH 152 or host 150 detect an unusually low HRV and forward the ECG waveform to an FDA-approved cloud cardiac service with automated expert ECG waveform analytics for detecting arrhythmias, atrial fibrillation, and other conditions. The results reveal that the elderly user has an underlying heart condition that requires further investigation.

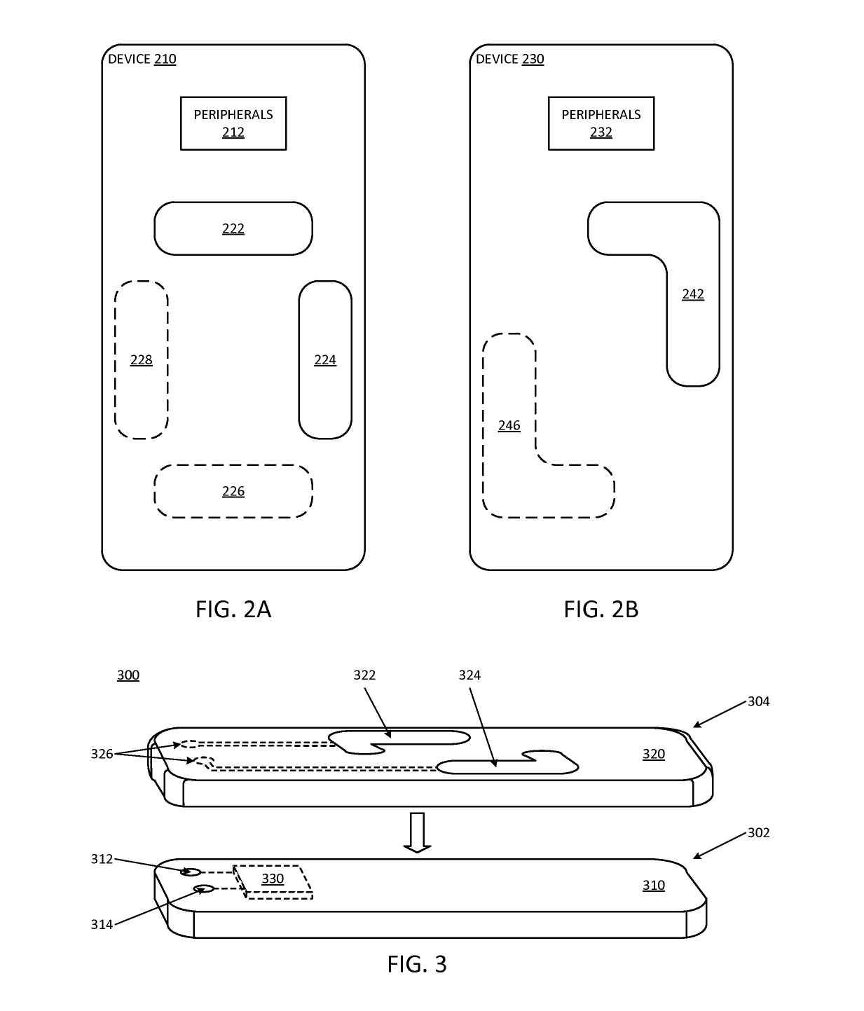

FIG. 2A is a block diagram of an embodiment of a computing device with strategically placed ECG electrodes for opportunistic heart rate monitoring. Device 210 is one example of a handheld device in accordance with device 110 of system 100. Device 210 is illustrated from a perspective of looking at a face the device, namely the back face of the device. The device back face may be flat or curved. In one embodiment, device 210 includes peripherals 212 on the back face. Peripherals 212 can include a camera, an LED flash, and/or other sensors.

Electrodes 222, 224, 226, and 228 are strategically located on device 210 to increase the frequency of simultaneous left and right electrode contact with the left and right fingers or thumbs, respectively, during active use of the device. The likelihood the user will make contact with the electrodes can be similar when device 210 is held in portrait (the display is taller than it is wide) and landscape (the display is wider than it is tall) mode. Device 210 can monitor an observable ECG waveform independent of how the device is oriented.

In one embodiment, electrodes 222 and 224 can be considered “Left” electrodes, and electrodes 226 and 228 can be considered “Right” electrodes. In one embodiment, 222 and 224 are jointly considered one electrode, even though they are separate surfaces, since they connect to the same contact of the internal ECG circuit (not explicitly shown). While two contact surfaces are illustrated for each hand, in one embodiment, the number of surfaces for each hand can be increased. Any combination of contact surfaces can be used with each other as long as one of 222 and 224 (the “solid line” electrodes) is contacted with one hand, and one of 226 and 228 (the “dashed line” electrodes) is contacted with the other hand.

It will be understood that with reference to the face of the surface of device 210 as illustrated, the dashed line electrodes and the solid line electrodes can be considered to be on opposite sides of the face of the body of device 210. Electrodes 222 and 226 are coupled to different inputs of an internal ECG circuit, and are on opposite sides of the face from each other. If the device face is considered to be split along a diagonal running between the solid line electrodes and the dashed line electrodes, any solid line electrode can be considered on an opposite side of the body of the device from any dashed line electrode.

FIG. 2B is a block diagram of an embodiment of a computing device with strategically placed ECG electrodes for opportunistic heart rate monitoring. Device 230 is one example of a handheld device in accordance with device 110 of system 100. Device 230 is illustrated from a perspective of looking at a face the device, namely the back face of the device. The device back face may be flat or curved. In one embodiment, device 230 includes peripherals 232 on the back face. Peripherals 232 can include a camera, an LED flash, and/or other sensors.

Electrode 242 is considered opposite electrode 246 on the face of device 230, and electrode 242 and electrode 246 connect to different contacts on an internal ECG circuit (not explicitly shown). The electrode design of device 230 can be considered to coalesce the two R electrodes and the two L electrodes into L-shaped electrodes while retaining the observability of the ECG waveform in portrait and landscape modes. Electrodes 242 and 246 can be considered to have horizontal and vertical portions, as each includes a portion that extends into the x-dimension and y-dimension for the face of device 230.

In one embodiment, electrodes 242 and/or 246 can be strips of conductive surface. The electrodes can rounded, squared, and even set at angles. The electrodes can be placed with x and y orientations that are offset relative to x and y orientations of the face of device 230, as long as there are electrodes on opposite sides to enable opportunistic contact by a user. Thus, the shapes shown are merely one of many possible examples. The illustrations are not limiting to the limitless combinations of shapes in which the ECG electrodes can be integrated onto the body of the handheld computing devices 210 or 230.

FIG. 3 is a block diagram of an embodiment of a device cover having strategically placed ECG electrodes for opportunistic heart rate monitoring. System 300 is one example of a handheld device in accordance with device 110 of system 100. System 300 is one example of a handheld device in accordance with device 210 of FIG. 2A or device 230 of FIG. 2B. For simplicity, the electrode shape shown in system 300 is L-shaped electrodes, but such an illustration is not limiting.

In one embodiment, system 300 includes the handheld computing device 302 and cover 304. Face 310 of computing device 302 is the surface that includes contacts 312 and 314. Contacts 312 and 314 represent contacts in the external or user-facing face 310. Contacts 312 and 314 represent contact points in the housing of device 310. Internal ECG circuit 330 is within device 302. It will be understood that the components in system 300 are not necessarily to scale. Internal ECG circuit 330 is an ECG circuit in accordance with any embodiment described herein. Circuit 330 is connected to contacts 312 and 314. In one embodiment, contacts 312 and 314 would connect directly or would be electrodes on the surface of device 302. As illustrated, contacts 312 and 314 connect electrically to contacts on cover 304. Cover 304 surrounds face 310 of the housing of device 302.

Cover 304 includes face 320, on which is located electrodes 322 and 324. Electrodes 322 and 324 connect, respectively, to contacts 312 and 314 via electrical points 326. When cover 304 is placed on device 302, system 300 includes ECG electrodes strategically placed for opportunistic contact by a user that uses device 302. Closed loop contact by the user (two-handed contact, one hand per electrode) enables ECG circuit 330 to opportunistically monitor HR information for the user. In one embodiment, ECG circuit 330 provides HR information for integration with other sensor information to provide contextual HR monitoring for user contact across electrodes 322 and 324.

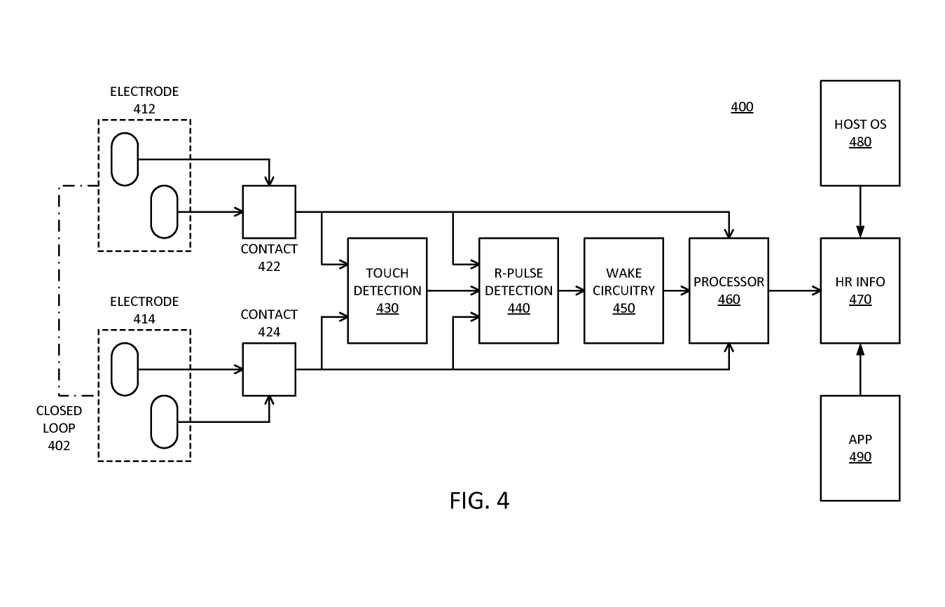

FIG. 4 is a block diagram of an embodiment of a system that performs opportunistic monitoring including detecting whether user contact is two-handed or one-handed. System 400 represents one embodiment of an ECG circuit in accordance with any embodiment described herein, such as ECG 140 of system 100. Electrodes 412 and 414 are integrated into the body or housing of a computing device. Electrodes 412 and 414 can include Left and Right electrodes and be differential electrodes. They are positioned strategically in accordance with any embodiment described herein to facilitate opportunistic contact by the user. When contact by the user simultaneously touches both electrodes 412 and 414, closed loop 402 is formed.

In one embodiment, all conductive surfaces of electrode 412 connect to contact 422, which can be in the body of the computing device, or can be an internal point connected to inputs of an ECG AFE and controller. Similarly, in one embodiment, all conductive surfaces of electrode 414 connect to contact 424, which can be in the body of the computing device, or can be an internal point connected to inputs of an ECG AFE and controller.

In one embodiment, touch detection 430 represents an ECG differential input of an ECG controller. When closed loop 402 forms, the differential input circuit impedance changes. Such a change in impedance can be used to detect if a user is touching the electrodes. Traditionally, contact detection alone has been used to wake “downstream” circuitry such as the signal processing and communication/transmission hardware. Thus, when touch detection 430 detects closed loop 402 across electrodes 412 and 414, traditionally system 400 would wake up processor 460 and process the input signals. However, when monitoring for opportunistic user contact, closed loop 402 may or may not provide a valid ECG input signal. Thus, in one embodiment, system 400 includes one or more components to determine if the closed loop results in a valid signal to monitor.

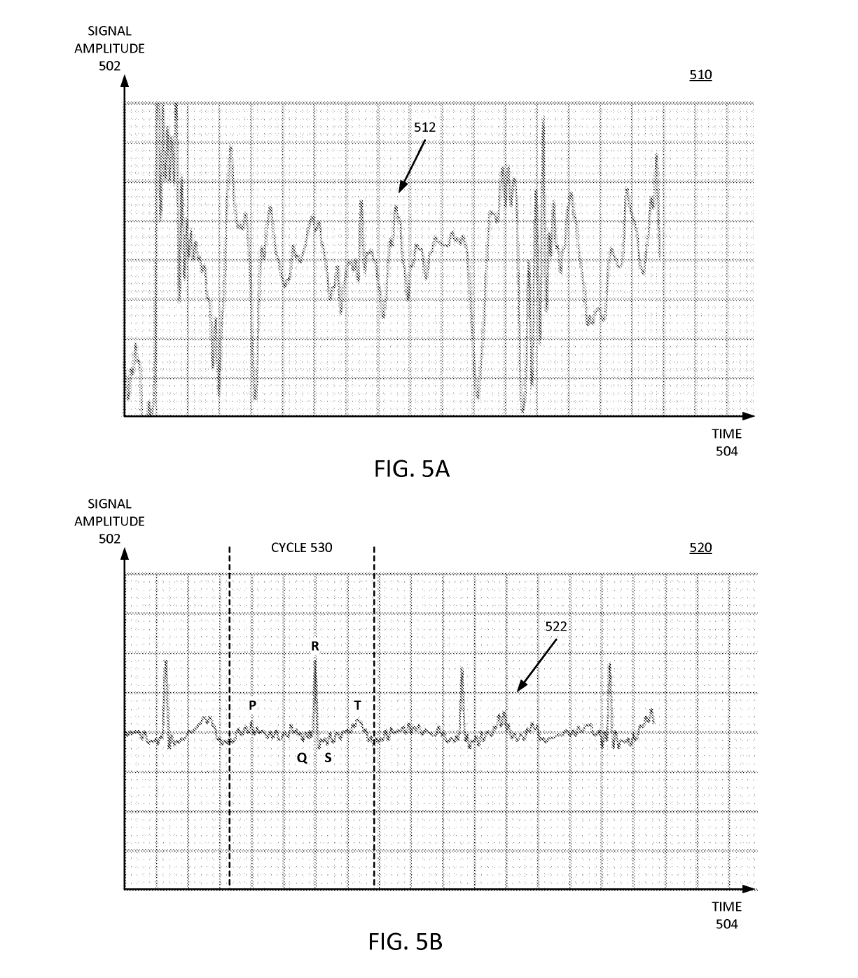

In one embodiment, system 400 includes R-pulse detection 440. While a specific R-pulse detection is illustrated, other signal detection methods such as impedance level detection, could be used in addition to or as an alternative to R-pulse detection. Reference to R-pulse detection refers to the so-called “PQRST” waveform of a heart beat input signal. Consider the characteristic PQRST signal pattern of FIG. 5B which illustrates a valid ECG input resulting from a two-handed closed loop versus the noise pattern of FIG. 5A which illustrates noise from a one-handed closed loop.

In one embodiment, R-pulse detection 440 triggers wake circuit 450 to wake up processor 460 (and communication hardware and logic, not shown) only when a repeated pattern of ‘R’ peaks is detected. When electrodes 412 and 414 are touched and closed loop 402 detected, in one embodiment, the resulting impedance change triggers R-pulse detection module 440 to analyze a differential electrode input signal for repeated R pulses found in a typical ECG waveform. The repeated R pulses are a series of signal peaks that have much higher amplitude than the rest of the signal, and occur in a regular period. When the signature pattern of R pulses is detected, R-pulse detection 440 can enable wake circuitry 450.

In one embodiment, system 400 simply measures input impedance, and relies on the fact that two-handed and one-handed closed loops have different characteristic input impedance. It will be understood that impedance detection can detect when the input transitions from an open loop (infinite impedance) to a finite impedance. Thus, system 400 can include thresholds of input impedance ranges (for example, in an impedance detection module, not shown), and determine whether the input is within a range (e.g., range of finite input impedances) associated with two-handed input or one-handed input. R-pulse detection 440 may be more accurate than simple impedance checking, but different false signal rejection can be used in different implementations (e.g., use R-pulse detection in one implementation, and impedance detection in another implementation).

In one embodiment, system 400 includes an input impedance mechanism with predetermined ranges, as described above. In one embodiment, system 400 includes input detection to determine whether the input has a pattern of an EMG (electromyograph) signal. In one embodiment, system 400 can include a detection module or detection circuit that detects skeletal muscle signaling on an input of electrodes 412 and 414. EMG signals contrast to ECG signals, as they are produced by the skeletal muscles instead of the electrical pattern of the heart activity. Thus, system 400 could determine that an input signal has a pattern similar to an EMG signal, and not perform heart rate monitoring when the wrong signal appears on the inputs of electrodes 412 and 414.

Any mechanism that waits to wake up processor 460 until a valid ECG input is detected can improve power performance by keeping downstream mixed signal and/or digital components in sleep mode until a bona fide ECG signal is detected. In one embodiment, system 400 includes touch detection 430 and a touch type detection module (such as R-pulse detection or impedance detection), which are always on. Wake circuitry 450, processor 460, and any communication circuitry can be disabled until a valid input is detected.

Processor 460 processes input ECG signal data. Processor 460 generates HR information 470, which is stored in system 400. HR information 470 can be accessed by host OS (operating system) 480 and/or applications 490 executing under host OS 480. Applications 490 can be applications provided by the manufacturer of the mobile device and/or by ISVs (independent software vendors). In one embodiment, processor 460 provides HR information 470 as a platform service, and thus can be available in the background of a computing device without a user needing to load a specific application. Host OS 480 can apply contextual HR information as a service to provide alerts or other functions of the mobile device. Other applications 490 can also be enabled to access and use contextual HR information for other functionality, such as integration with other health monitoring equipment.

One consideration for input detection for system 400 is the emergence of wireless charging for smartphones or other handheld electronics. If a mobile or handheld device including system 400 includes conductive electrodes on a body of the device, it will be understood that use of wireless charging could potentially short out the inputs. In one embodiment, touch detection 430 or other module in system 400 includes short detection circuitry to determine that closed loop 402 is a short circuit that provides power into the inputs. In one embodiment, such short detection can trigger system 400 to not only keep the downstream modules in a low power state, but transition AFE components to a high impedance input state or otherwise disables an input to prevent damage to the circuits. In one embodiment, system 400 can receive a signal from a wireless charging detection system, and initiate an input protection state in response to such a signal in addition to, or as an alternative to, initiating input protection based on detecting a short.

FIG. 5A is a diagrammatic representation of an embodiment of a one-handed ECG signal. Diagram 510 illustrates input signal or waveform 512, which might be received as an input to ECG electrodes on a handheld device during opportunistic monitoring. Input signal 512 is an illustration of signal amplitude 502 received over time 504. Basic input impedance detection can be triggered by one hand of the user spanning both ECG electrodes at the same time. The waveform of input signal 512 is not an ECG waveform since the circuit is not across the user's heart (both left and right hands are required to complete the circuit across the heart). Input signal 512 is typical of an input signal received by touching both electrodes with one hand, and does not look like an actual ECG signal. Thus, input signal 512 can be detected as a false input signal, produced by one-handed electrode activation.

FIG. 5B is a diagrammatic representation of an embodiment of a two-handed ECG signal. Diagram 520 illustrates input signal or waveform 522. Input signal 522 can be received as an input to ECG electrodes on a handheld device during opportunistic monitoring. Input signal 522 illustrates a waveform showing signal amplitude 502 versus time 504. It will be understood that diagram 520 has the same scale as diagram 510, and thus the axes are labeled the same.

Diagram 520 shows cycle 530, which is a unit of a repeated of cycle of heart activity recorded in the signal. It will be seen that cycle 530 repeats throughout input signal 522. The labels of cycle 530 include ‘P’ which represents a small peak of the atrial contraction, ‘Q’ which represents the leading valley in the contraction of the ventricles, ‘R’ which represents the primary peak of the contraction of the ventricles, ‘S’ which represents the trailing valley in the contraction of the ventricles, and ‘T’ which represents the small peak of the relaxation of the ventricles. The R-peak tends to be orders of magnitude larger than the other features. When such a signal is detected, the ECG circuit can identify the signal as valid and cause the ECG processor to process and log the signal.

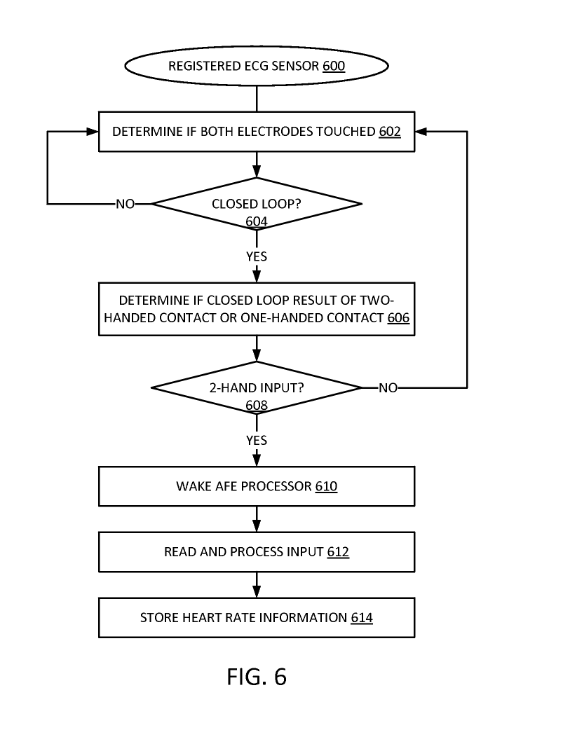

FIG. 6 is a flow diagram of an embodiment of a process for monitoring an ECG input. Process 600 is a monitoring process for a registered ECG sensor. In one embodiment, a handheld device includes an AFE of an ECG circuit that has hardware to determine if both electrodes are opportunistically touched by the user, 602. If there is not a closed loop across the differential electrodes, 604 NO branch, the ECG circuit continues to monitor the input to the electrodes, 602. If there is a closed loop across the electrodes, 604 YES branch, in one embodiment, the ECG circuit determines if the closed loop is the result of two-handed contact or one-handed contact, 606.

If the ECG circuit determines that the closed loop is the result of one-handed contact, 608 NO branch, the ECG circuit continues to monitor the input to the electrodes, 602. Thus, the ECG circuit can filter inputs to process only two-handed activation of the electrodes. If the input is the result of two-handed input, 608 YES branch, in one embodiment, a wake circuit wakes the processor and communication circuits, 610. The processor can then read and process the input, 612. The processor causes the heart rate information to be recorded, 614. In one embodiment, the processor sends the processed input information to a sensor hub that can aggregate sensor information and provide contextual HR information.

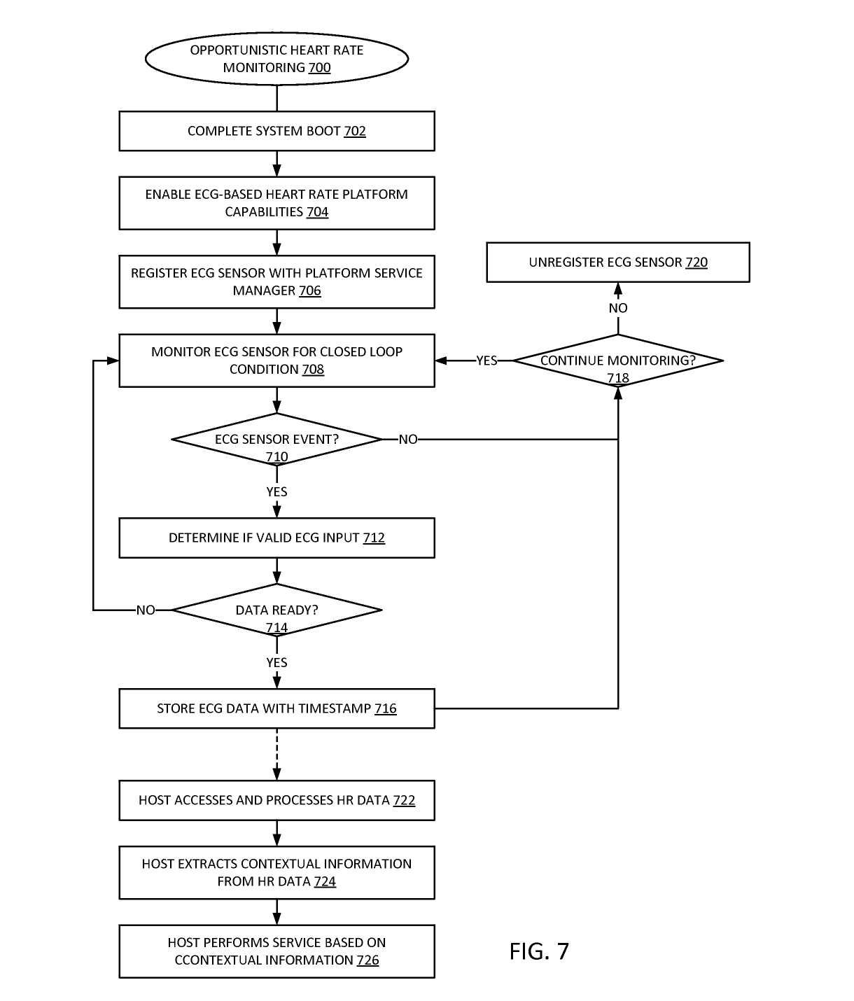

FIG. 7 is a flow diagram of an embodiment of a process for opportunistic heart rate monitoring. Process 700 enables an ECG circuit to perform opportunistic heart rate monitoring on a mobile/handheld device via integrated ECG electrodes. In one embodiment, the device in which the ECG circuit or ECG subsystem is incorporated completes its boot process, 702. The boot process loads the host operating system and enables the hardware and software platforms for the device. In one embodiment, the system boot includes enabling ECG-based heart rate platform capabilities, 704. The ECG subsystem can be managed by a platform service and/or an application running in a background of a mobile device. The service can provide access to HR information to the platform, including other applications executing on the platform.

In one embodiment, the ECG sensor registers with the platform service manager, 706. The ECG subsystem is then enabled to monitoring the ECG sensor for a closed loop condition across the integrated ECG electrodes. The ECG electrodes can be in accordance with any embodiment described herein, and are strategically placed on the device to allow opportunistic contact of both electrodes with opposite hands by a user of the device. If the ECG subsystem detects an ECG sensor event, 710 YES branch, in one embodiment, the ECG subsystem determines if the sensor event is a valid ECG input, 712. An ECG sensor event occurs when both electrodes are touched by the user to create a closed loop. In one embodiment, the ECG sensor event only results in processing the input if the data is a valid ECG signal.

The AFE of the ECG subsystem detects input impedance changes when there is a closed loop across the ECG electrodes. Input impedance for single hand activation is likely different, perhaps lower, than when both hands activate the input. Thus, in one embodiment, the AFE includes input impedance detection and determines whether the input impedance is within an expected, predetermined range typical for a valid ECG signal. Such predetermination can be the result of training the sensor and subsystem, for example. In one embodiment, the AFE includes R-peak or R-pulse detection. The R peak is narrow and has the largest amplitude. In one embodiment, the AFE can detect R pulses, and trigger wake circuitry only when such recurring peaks are detected. In one embodiment, the AFE can also perform short circuit detection to shut down inputs to the ECG subsystem if the device is placed on a mechanism that performs wireless battery charging.

If the signal is not a valid ECG input, 714 NO branch, the input does not represent data ready to log, and the ECG subsystem can continue to monitor the ECG sensor for a sensor event, 708. If the signal is a valid ECG input, 714 YES branch, the ECG subsystem can store the ECG data or HR information, 716. In one embodiment, the ECG subsystem logs the ECG data with timestamp information, which can help is generating contextual HR information.

If there is not an ECG sensor event, 710 NO branch, in one embodiment, the ECG subsystem can determine whether or not to continue monitoring for ECG inputs, 718. For example, the subsystem can check periodically for inputs, and stop monitoring if one is not detected. In another example, other sensor information can trigger the ECG subsystem to stop monitoring for a period of time, and initiate monitoring at some later time, such as during certain hours of the day, or after the device sits idle for a period of time. Thus, in one embodiment, the ECG subsystem will only monitor for opportunistic ECG contact when sensor input indicates that the device is “in use” by the user, and may shut down otherwise. If the ECG subsystem is to continue monitoring, 718 YES branch, it continues to monitor for an ECG sensor event, 708. If the ECG subsystem is to discontinue monitoring, 718 NO branch, in one embodiment the subsystem can unregister the ECG sensor, 720.

In one embodiment, after storing the ECG data, the system host can access the data for contextual use. In one embodiment, the host accesses and processes the HR data, 722. For example, the ECG subsystem may transmit the HR data to a sensor hub or other processing component of the host. In one embodiment, the host extracts contextual information from the HR data, 724, such as by combining HR data with data from other system sensors. In one embodiment, extracting contextual information can include accessing a cloud-based service and exchanging data with the cloud service. In one embodiment, the host performs a service based on contextual HR information, 726. The service can be in accordance with any embodiment described herein, where the mobile device can generate a message to a user and/or to medical professionals.

As mentioned above, the HR data can be time stamped and kept in a history database. In one embodiment, in addition to timestamped HR data, the computing device can record information about the user's activity (e.g., walking, running, climbing stairs, or other activity) and/or the user's environment (e.g., cold, hot, or other information). The additional information can also be timestamped. A service can analyze all data, including correlating the data by timestamp, and provide reports based on the analysis. The reports can be graphical and/or textual. In one embodiment, if HR data ranges match the context, then there is no need to generate an alert. Thus, if the HR or HRV is within an expected range for the activity and environment inferred from additional sensor data, there is not an alert condition. However, if at time X, HR data was elevated when other sensors indicated that the user was sedentary, it could be an alert condition. The alerting could be real-time and/or part of daily or weekly report.

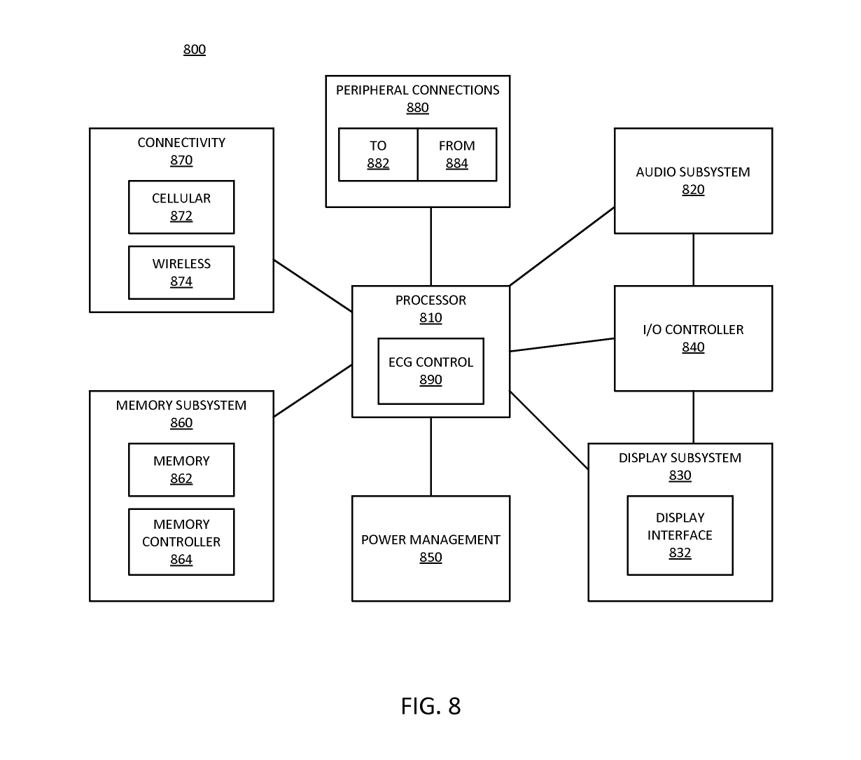

FIG. 8 is a block diagram of an embodiment of a mobile device in which opportunistic heart rate monitoring can be implemented. Device 800 represents a mobile computing device, such as a computing tablet, a mobile phone or smartphone, a wireless-enabled e-reader, wearable computing device, or other mobile device. It will be understood that certain of the components are shown generally, and not all components of such a device are shown in device 800.

Device 800 includes processor 810, which performs the primary processing operations of device 800. Processor 810 can include one or more physical devices, such as microprocessors, application processors, microcontrollers, programmable logic devices, or other processing means. The processing operations performed by processor 810 include the execution of an operating platform or operating system on which applications and/or device functions are executed. The processing operations include operations related to I/O (input/output) with a human user or with other devices, operations related to power management, and/or operations related to connecting device 800 to another device. The processing operations can also include operations related to audio I/O and/or display I/O.

In one embodiment, device 800 includes audio subsystem 820, which represents hardware (e.g., audio hardware and audio circuits) and software (e.g., drivers, codecs) components associated with providing audio functions to the computing device. Audio functions can include speaker and/or headphone output, as well as microphone input. Devices for such functions can be integrated into device 800, or connected to device 800. In one embodiment, a user interacts with device 800 by providing audio commands that are received and processed by processor 810.

Display subsystem 830 represents hardware (e.g., display devices) and software (e.g., drivers) components that provide a visual and/or tactile display for a user to interact with the computing device. Display subsystem 830 includes display interface 832, which includes the particular screen or hardware device used to provide a display to a user. In one embodiment, display interface 832 includes logic separate from processor 810 to perform at least some processing related to the display. In one embodiment, display subsystem 830 includes a touchscreen device that provides both output and input to a user. In one embodiment, display subsystem 830 includes a high definition (HD) display that provides an output to a user. High definition can refer to a display having a pixel density of approximately 100 PPI (pixels per inch) or greater, and can include formats such as full HD (e.g., 1080p), retina displays, 4K (ultra high definition or UHD), or others.

I/O controller 840 represents hardware devices and software components related to interaction with a user. I/O controller 840 can operate to manage hardware that is part of audio subsystem 820 and/or display subsystem 830. Additionally, I/O controller 840 illustrates a connection point for additional devices that connect to device 800 through which a user might interact with the system. For example, devices that can be attached to device 800 might include microphone devices, speaker or stereo systems, video systems or other display device, keyboard or keypad devices, or other I/O devices for use with specific applications such as card readers or other devices.

As mentioned above, I/O controller 840 can interact with audio subsystem 820 and/or display subsystem 830. For example, input through a microphone or other audio device can provide input or commands for one or more applications or functions of device 800. Additionally, audio output can be provided instead of or in addition to display output. In another example, if display subsystem includes a touchscreen, the display device also acts as an input device, which can be at least partially managed by I/O controller 840. There can also be additional buttons or switches on device 800 to provide I/O functions managed by I/O controller 840.

In one embodiment, I/O controller 840 manages devices such as accelerometers, cameras, light sensors or other environmental sensors, gyroscopes, global positioning system (GPS), or other hardware that can be included in device 800. The input can be part of direct user interaction, as well as providing environmental input to the system to influence its operations (such as filtering for noise, adjusting displays for brightness detection, applying a flash for a camera, or other features). In one embodiment, device 800 includes power management 850 that manages battery power usage, charging of the battery, and features related to power saving operation.

Memory subsystem 860 includes memory device(s) 862 for storing information in device 800. Memory subsystem 860 can include nonvolatile (state does not change if power to the memory device is interrupted) and/or volatile (state is indeterminate if power to the memory device is interrupted) memory devices. Memory 862 can store application data, user data, music, photos, documents, or other data, as well as system data (whether long-term or temporary) related to the execution of the applications and functions of system 800. In one embodiment, memory subsystem 860 includes memory controller 864 (which could also be considered part of the control of system 800, and could potentially be considered part of processor 810). Memory controller 864 includes a scheduler to generate and issue commands to memory device 862.

Connectivity 870 includes hardware devices (e.g., wireless and/or wired connectors and communication hardware) and software components (e.g., drivers, protocol stacks) to enable device 800 to communicate with external devices. The external device could be separate devices, such as other computing devices, wireless access points or base stations, as well as peripherals such as headsets, printers, or other devices.

Connectivity 870 can include multiple different types of connectivity. To generalize, device 800 is illustrated with cellular connectivity 872 and wireless connectivity 874. Cellular connectivity 872 refers generally to cellular network connectivity provided by wireless carriers, such as provided via GSM (global system for mobile communications) or variations or derivatives, CDMA (code division multiple access) or variations or derivatives, TDM (time division multiplexing) or variations or derivatives, LTE (long term evolution—also referred to as “4G”), or other cellular service standards. Wireless connectivity 874 refers to wireless connectivity that is not cellular, and can include personal area networks (such as Bluetooth), local area networks (such as WiFi), and/or wide area networks (such as WiMax), or other wireless communication. Wireless communication refers to transfer of data through the use of modulated electromagnetic radiation through a non-solid medium. Wired communication occurs through a solid communication medium.

Peripheral connections 880 include hardware interfaces and connectors, as well as software components (e.g., drivers, protocol stacks) to make peripheral connections. It will be understood that device 800 could both be a peripheral device (“to” 882) to other computing devices, as well as have peripheral devices (“from” 884) connected to it. Device 800 commonly has a “docking” connector to connect to other computing devices for purposes such as managing (e.g., downloading and/or uploading, changing, synchronizing) content on device 800. Additionally, a docking connector can allow device 800 to connect to certain peripherals that allow device 800 to control content output, for example, to audiovisual or other systems.

In addition to a proprietary docking connector or other proprietary connection hardware, device 800 can make peripheral connections 880 via common or standards-based connectors. Common types can include a Universal Serial Bus (USB) connector (which can include any of a number of different hardware interfaces), DisplayPort including MiniDisplayPort (MDP), High Definition Multimedia Interface (HDMI), Firewire, or other type.

In one embodiment, system 800 includes ECG control 890, which can include an ECG subsystem or ECG circuit in accordance with any embodiment described herein. The ECG subsystem includes a connection to electrodes placed in a body of system 800 in accordance with any embodiment described herein. ECG control 890 includes signal detection and signal processing hardware. In one embodiment, ECG control 890 includes false ECG triggering detection, such as by impedance detection or input signal analysis (e.g., R-pulse detection).

In one aspect, a handheld computing device includes: a first ECG (electrocardiogram) contact integrated into the device to connect a first ECG electrode to an internal ECG circuit within the device; and a second ECG contact integrated into the device to connect a second ECG electrode to the internal ECG circuit within the device; wherein the first and second ECG electrodes have a vertical portion and a horizontal portion, wherein the first and second ECG electrodes are positioned on opposite sides of a face of a body of the device to enable opportunistic two-hand contact by a user of the device when the device is used in either landscape or portrait orientation; and wherein the internal ECG circuit is to detect two-hand contact by the user on the first and second electrodes, and perform ECG monitoring in response to detecting two-hand contact.

In one embodiment, the first and second ECG electrodes comprise electrodes integrated into a body of the device. In one embodiment, the first and second ECG electrodes comprise electrodes integrated into a body of a separate cover of the device, and connected to the contacts integrated into the body of the device. In one embodiment, the vertical portion and the horizontal portion comprise separate electrodes coupled to a common ECG contact. In one embodiment, the vertical portion and the horizontal portion comprise portions of an ‘L-shaped’ electrode coupled to the ECG contact. In one embodiment, the internal ECG circuit is to detect two-hand contact including detecting a finite impedance across the first and second electrodes, and determining that the finite impedance has a value within a range predetermined to indicate two-hand contact. In one embodiment, the internal ECG circuit is to detect two-hand contact including analyzing an input signal from the first and second electrodes to determine if the input signal has a PQRST pattern. In one embodiment, the internal ECG circuit further includes an electromyograph (EMG) circuit to detect skeletal muscle signaling on an input of the first and second electrodes, wherein when the EMG circuit detects skeletal muscle signaling on the input of the first and second electrodes, the internal ECG circuit does not perform heart rate monitoring. In one embodiment, the internal ECG circuit is to perform heart rate monitoring as a background process, including storing heart rate information for a host operating system of the device. In one embodiment, the device further including: an integrated environmental sensor to detect environmental information; and a processor to integrate heart rate information from the internal ECG circuit with the integrated environmental sensor. In one embodiment, the environmental sensor comprises one of multiple sensors, and further comprising: an integrated sensor hub to receive input from the multiple sensors, wherein the processor integrated heart rate information from the internal ECG circuit with data from the multiple sensors. In one embodiment, the environmental sensor comprises a motion detection sensor. In one embodiment, the internal ECG circuit further includes a short circuit detector to detect a low-resistance connection or short circuit between the first and second electrodes; wherein the internal ECG circuit is to disable an input in response to detecting a short circuit between the first and second electrodes.

In one aspect, a handheld computing device includes: a first ECG (electrocardiogram) contact integrated into the device to connect a first ECG electrode to an internal ECG circuit within the device; a second ECG contact integrated into the device to connect a second ECG electrode to the internal ECG circuit within the device; wherein the first and second ECG electrodes have a vertical portion and a horizontal portion, wherein the first and second ECG electrodes are positioned on opposite sides of a face of a body of the device to enable opportunistic two-hand contact by a user of the device when the device is used in either landscape or portrait orientation; and wherein the internal ECG circuit is to detect two-hand contact by the user on the first and second electrodes, and perform ECG monitoring in response to detecting two-hand contact; and logic executing on the device to connect to a cloud-based computing resource, wherein the logic is to provide heart rate information from the internal ECG circuit to the cloud-based computing resource and receive analysis information on the heart rate information from the cloud-based computing resource.

In one embodiment, the first and second ECG electrodes integrated into the body of the device comprise electrodes integrated directly into a housing of the device. In one embodiment, the first and second ECG electrodes integrated into the body of the device comprise electrodes integrated into a cover that surrounds the housing of the device. In one embodiment, the vertical portion and the horizontal portion comprise separate electrodes coupled to a common ECG contact. In one embodiment, the vertical portion and the horizontal portion comprise connected portions of an ‘L-shaped’ electrode coupled to the ECG contact. In one embodiment, the internal ECG circuit is to detect two-hand contact including detecting a finite impedance across the first and second electrodes having a value within a range predetermined to indicate two-hand contact. In one embodiment, the internal ECG circuit is to detect two-hand contact including analyzing an input signal from the first and second electrodes to determine if the input signal has a PQRST pattern. In one embodiment, the internal ECG circuit is to detect two-hand contact including detecting that an input signal on the first and second electrodes is not an electromyograph (EMG) signal. In one embodiment, the internal ECG circuit is to perform heart rate monitoring as a background process, including storing heart rate information for a host operating system of the device. In one embodiment, the device further including: an integrated sensor hub that uses environmental and motion detection sensors to infer user context and user environmental information; and a processor to integrate heart rate information from the internal ECG circuit with the user context and user environment data from the integrated sensor hub. In one embodiment, the internal ECG circuit further includes a short circuit detector to detect a low-resistance connection or short circuit between the first and second electrodes; wherein the internal ECG circuit is to disable an input in response to detecting a short circuit between the first and second electrodes.

In one aspect, a method for monitoring heart rate information includes: detecting a closed circuit connection to first and second ECG (electrocardiogram) contacts, wherein the first and second ECG contacts are ECG electrodes integrated into the body of a handheld electronic device and connected to an internal ECG circuit within the device, wherein the first and second ECG electrodes have a vertical portion and a horizontal portion, and wherein the first and second ECG electrodes are positioned on opposite sides of a face of the body of the device to enable opportunistic two-hand contact by a user of the device when the device is used in either landscape or portrait orientation; and performing ECG monitoring of an input signal from the first and second ECG electrodes in response to detecting the closed circuit connection.

In one embodiment, the first and second ECG electrodes comprise electrodes integrated into a body of a separate cover of the device, and connected to the contacts integrated into the body of the device. In one embodiment, the vertical portion and the horizontal portion comprise separate electrodes coupled to a common ECG contact. In one embodiment, the vertical portion and the horizontal portion comprise portions of a continuous, L-shaped electrode coupled to an ECG contact. In one embodiment, detecting the closed circuit connection to first and second ECG contacts further comprises detecting a finite impedance across the first and second electrodes, and determining that the finite impedance has a value within a range predetermined to indicate two-hand contact. In one embodiment, detecting the closed circuit connection to first and second ECG contacts further comprises receiving an input signal from the first and second electrodes, and detecting a PQRST pattern in the input signal.

In one embodiment, detecting the closed circuit connection to first and second ECG contacts further comprises detecting an input signal from the first and second electrodes, and determining that the input signal is different from an electromyograph (EMG) signal based on the input signal. In one embodiment, performing ECG monitoring comprises performing heart rate monitoring as a background process, including storing heart rate information for a host operating system of the device. In one embodiment, further comprising: integrating heart rate information from the internal ECG circuit with environmental sensor information from an integrated environmental sensor on the device. In one embodiment, integrating heart rate information with environmental sensor information comprises integrating heart rate information with environmental sensor in an integrated sensor hub of the handheld electronic device. In one embodiment, integrating heart rate information with environmental sensor information comprises integrating heart rate information with data from the multiple integrated environmental sensors. In one embodiment, integrating heart rate information with environmental sensor information comprises integrating heart rate information with data from a motion detection sensor. In one embodiment, further comprising: detecting a low-resistance connection or short circuit between the first and second electrodes; and disabling an input in response to detecting the low-resistance connection or short circuit between the first and second electrodes.

In one aspect, an article of manufacture comprising a computer readable storage medium having content stored thereon, which when accessed causes a machine to perform operations for monitoring heart rate information, including: detecting a closed circuit connection to first and second ECG (electrocardiogram) contacts, wherein the first and second ECG contacts are ECG electrodes integrated into the body of a handheld electronic device and connected to an internal ECG circuit within the device, wherein the first and second ECG electrodes have a vertical portion and a horizontal portion, and wherein the first and second ECG electrodes are positioned on opposite sides of a face of the body of the device to enable opportunistic two-hand contact by a user of the device when the device is used in either landscape or portrait orientation; and performing ECG monitoring of an input signal from the first and second ECG electrodes in response to detecting the closed circuit connection. The article of manufacture can include content for performing operations in accordance with any embodiment of the method for monitoring heart rate information set forth above.

In one aspect, an apparatus for monitoring heart rate information includes: means for detecting a closed circuit connection to first and second ECG (electrocardiogram) contacts, wherein the first and second ECG contacts are ECG electrodes integrated into the body of a handheld electronic device and connected to an internal ECG circuit within the device, wherein the first and second ECG electrodes have a vertical portion and a horizontal portion, and wherein the first and second ECG electrodes are positioned on opposite sides of a face of the body of the device to enable opportunistic two-hand contact by a user of the device when the device is used in either landscape or portrait orientation; and means for performing ECG monitoring of an input signal from the first and second ECG electrodes in response to detecting the closed circuit connection. The apparatus can include means for performing operations in accordance with any embodiment of the method for monitoring heart rate information set forth above.

Flow diagrams as illustrated herein provide examples of sequences of various process actions. The flow diagrams can indicate operations to be executed by a software or firmware routine, as well as physical operations. In one embodiment, a flow diagram can illustrate the state of a finite state machine (FSM), which can be implemented in hardware and/or software. Although shown in a particular sequence or order, unless otherwise specified, the order of the actions can be modified. Thus, the illustrated embodiments should be understood only as an example, and the process can be performed in a different order, and some actions can be performed in parallel. Additionally, one or more actions can be omitted in various embodiments; thus, not all actions are required in every embodiment. Other process flows are possible.

To the extent various operations or functions are described herein, they can be described or defined as software code, instructions, configuration, and/or data. The content can be directly executable (“object” or “executable” form), source code, or difference code (“delta” or “patch” code). The software content of the embodiments described herein can be provided via an article of manufacture with the content stored thereon, or via a method of operating a communication interface to send data via the communication interface. A machine readable storage medium can cause a machine to perform the functions or operations described, and includes any mechanism that stores information in a form accessible by a machine (e.g., computing device, electronic system, etc.), such as recordable/non-recordable media (e.g., read only memory (ROM), random access memory (RAM), magnetic disk storage media, optical storage media, flash memory devices, etc.). A communication interface includes any mechanism that interfaces to any of a hardwired, wireless, optical, etc., medium to communicate to another device, such as a memory bus interface, a processor bus interface, an Internet connection, a disk controller, etc. The communication interface can be configured by providing configuration parameters and/or sending signals to prepare the communication interface to provide a data signal describing the software content. The communication interface can be accessed via one or more commands or signals sent to the communication interface.

Various components described herein can be a means for performing the operations or functions described. Each component described herein includes software, hardware, or a combination of these. The components can be implemented as software modules, hardware modules, special-purpose hardware (e.g., application specific hardware, application specific integrated circuits (ASICs), digital signal processors (DSPs), etc.), embedded controllers, hardwired circuitry, etc.

Besides what is described herein, various modifications can be made to the disclosed embodiments and implementations of the invention without departing from their scope. Therefore, the illustrations and examples herein should be construed in an illustrative, and not a restrictive sense. The scope of the invention should be measured solely by reference to the claims that follow.

What is claimed is:

1. A handheld computing device, comprising:
a first ECG (electrocardiogram) contact integrated into the device to connect a first ECG electrode to an internal ECG circuit within the device; and
a second ECG contact integrated into the device to connect a second ECG electrode to the internal ECG circuit within the device;
wherein the first and second ECG electrodes have a vertical portion and a horizontal portion, wherein the first and second ECG electrodes are positioned on opposite sides of a face of a body of the device to enable opportunistic two-hand contact by a user of the device when the device is used in either landscape or portrait orientation; and
wherein the internal ECG circuit is to detect two-hand contact by the user on the first and second electrodes, and perform ECG monitoring in response to detecting two-hand contact.

2. The handheld computing device of claim 1, wherein the first and second ECG electrodes comprise electrodes integrated into a body of the device.

3. The handheld computing device of claim 1, wherein the first and second ECG electrodes comprise electrodes integrated into a body of a separate cover of the device, and connected to the contacts integrated into the body of the device.

4. The handheld computing device of claim 1, wherein the vertical portion and the horizontal portion comprise separate electrodes coupled to a common ECG contact.

5. The handheld computing device of claim 1, wherein the vertical portion and the horizontal portion comprise portions of an ‘L-shaped’ electrode coupled to the ECG contact.

6. The handheld computing device of claim 1, wherein the internal ECG circuit is to detect two-hand contact including detecting a finite impedance across the first and second electrodes, and determining that the finite impedance has a value within a range predetermined to indicate two-hand contact.

7. The handheld computing device of claim 1, wherein the internal ECG circuit is to detect two-hand contact including analyzing an input signal from the first and second electrodes to determine if the input signal has a PQRST pattern.

8. The handheld computing device of claim 1, wherein the internal ECG circuit further includes an electromyograph (EMG) circuit to detect skeletal muscle signaling on an input of the first and second electrodes, wherein when the EMG circuit detects skeletal muscle signaling on the input of the first and second electrodes, the internal ECG circuit does not perform heart rate monitoring.

9. The handheld computing device of claim 1, wherein the internal ECG circuit is to perform heart rate monitoring as a background process, including storing heart rate information for a host operating system of the device.

10. The handheld computing device of claim 1, the device further including:
an integrated environmental sensor to detect environmental information; and
a processor to integrate heart rate information from the internal ECG circuit with the integrated environmental sensor.

11. The handheld computing device of claim 10, wherein the environmental sensor comprises one of multiple sensors, and further comprising:
an integrated sensor hub to receive input from the multiple sensors, wherein the processor integrated heart rate information from the internal ECG circuit with data from the multiple sensors.

12. The handheld computing device of claim 10, wherein the environmental sensor comprises a motion detection sensor.

13. The handheld computing device of claim 1, wherein the internal ECG circuit further includes a short circuit detector to detect a low-resistance connection between the first and second electrodes; wherein the internal ECG circuit is to disable an input in response to detecting a short circuit between the first and second electrodes.

14. A handheld computing device, comprising:
a first ECG (electrocardiogram) contact integrated into the device to connect a first ECG electrode to an internal ECG circuit within the device;
a second ECG contact integrated into the device to connect a second ECG electrode to the internal ECG circuit within the device;
wherein the first and second ECG electrodes have a vertical portion and a horizontal portion, wherein the first and second ECG electrodes are positioned on opposite sides of a face of a body of the device to enable opportunistic two-hand contact by a user of the device when the device is used in either landscape or portrait orientation; and
wherein the internal ECG circuit is to detect two-hand contact by the user on the first and second electrodes, and perform ECG monitoring in response to detecting two-hand contact; and
logic executing on the device to connect to a cloud-based computing resource, wherein the logic is to provide heart rate information from the internal ECG circuit to the cloud-based computing resource and receive analysis information on the heart rate information from the cloud-based computing resource.

15. The handheld computing device of claim 14, wherein the first and second ECG electrodes integrated into the body of the device comprise electrodes either integrated directly into a housing of the device, or integrated into a cover that surrounds the housing of the device.

16. The handheld computing device of claim 14, wherein the vertical portion and the horizontal portion comprise either separate electrodes coupled to a common ECG contact or connected portions of an ‘L-shaped’ electrode coupled to the ECG contact.

17. The handheld computing device of claim 14, wherein the internal ECG circuit is to detect two-hand contact including one or more of: detecting a finite impedance across the first and second electrodes having a value within a range predetermined to indicate two-hand contact; analyzing an input signal from the first and second electrodes to determine if the input signal has a PQRST pattern; or, detecting that an input signal on the first and second electrodes is not an electromyograph (EMG) signal.

18. The handheld computing device of claim 14, the device further including:
an integrated sensor hub that uses environmental and motion detection sensors to infer user context and user environmental information; and
a processor to integrate heart rate information from the internal ECG circuit with the user context and user environment data from the integrated sensor hub.

19. The handheld computing device of claim 14, wherein the internal ECG circuit further includes a short circuit detector to detect a low-resistance connection between the first and second electrodes; wherein the internal ECG circuit is to disable an input in response to detecting a short circuit between the first and second electrodes.

20. A method for monitoring heart rate information, comprising:
detecting a closed circuit connection to first and second ECG (electrocardiogram) contacts,
wherein the first and second ECG contacts are ECG electrodes integrated into the body of a handheld electronic device and connected to an internal ECG circuit within the device,
wherein the first and second ECG electrodes have a vertical portion and a horizontal portion, and
wherein the first and second ECG electrodes are positioned on opposite sides of a face of the body of the device to enable opportunistic two-hand contact by a user of the device when the device is used in either landscape or portrait orientation; and
performing ECG monitoring of an input signal from the first and second ECG electrodes in response to detecting the closed circuit connection.

21. The method of claim 20, wherein the vertical portion and the horizontal portion comprise portions of a continuous, L-shaped electrode coupled to an ECG contact.

22. The method of claim 20, wherein detecting the closed circuit connection to first and second ECG contacts further comprises one or more of:
detecting a finite impedance across the first and second electrodes, and determining that the finite impedance has a value within a range predetermined to indicate two-hand contact;
receiving an input signal from the first and second electrodes, and detecting a PQRST pattern in the input signal; or
detecting an input signal from the first and second electrodes, and determining that the input signal is different from an electromyograph (EMG) signal based on the input signal.

23. The method of claim 20, further comprising:
integrating heart rate information from the internal ECG circuit with environmental sensor information from an integrated environmental sensor on the device.
